(19) United States
(12) Patent Application Publication
(10) Pub. No.: US 2005/0042207 A1
(43) Pub. Date: Feb. 24, 2005

(54) Stable liquid probiotic composition, preparation and applications thereof

(75) Inventor: Nellie Kelner-Padalka, Rishon LeZion (IL)

Correspondence Address: Martin Moynihan;c/o ANTHONY CASTORINA, SUITE 207, 2001 JEFFERSON DAVIS HIGHWAY, ARLINGTON, VA, 22202, US

(73) Assignee: The Bio Balance Corporation

(21) Appl. No.: 10/918,430

(22) Filed: Aug. 16, 2004

Related U.S. Application Data
(60) Provisional application No. 60/495,714, filed on Aug. 18, 2003.

(51) Int. Cl. A61K 45/00

(52) U.S. Cl. 424/93.4; 424/93.45

(57) ABSTRACT

A method for preparing liquid probiotic composition, comprising bacteria having at least a basal biologic activity, wherein said bacteria have been selected according to at least one selection pressure, wherein the composition preferably includes an autolysate (complete substances for maintaining bacteria) and wherein the composition is substantially free from substances suitable for bacterial growth but not similarly suitable for mammals, and particularly not suitable for human beings. Peptone and buffering salts, particularly phosphates, may not be harmful in small doses, but they are not specifically suitable for human beings and free from substances generated by bacteria.

This application claims the benefit of priority from 60/495,714, filed Aug. 18, 2003.

### FIELD OF THE INVENTION

The present invention relates to probiotic compositions, and more particularly to a method of preparing a liquid probiotic composition comprising a non-pathogenic bacterial strain which may be stored in biologically active form for long periods of time, as well as compositions and methods of treatment thereof.

### BACKGROUND OF THE INVENTION

Probiotic bacteria are those which are beneficial to humans and/or animals. The use of probiotic bacteria is known in the art for improving the microbial balance in the intestinal tract of mammals, in order to prevent or treat gastro-enteric infections and other diseases or disorders involving and/or causing changes in or to the intestinal microflora composition, and/or resulting in any change to the microflora composition, and/or maintaining such changes, as well as changes to the microflora composition which actively cause or potentiate such diseases or disorders.

However, the results of studies carried out to date have been inconsistent and/or ambiguous. For example, in some studies, the use of probiotic bacteria alone to treat “traveler's diarrhea” was not sufficient to provide a significant effect in patients as opposed to placebo, yet the combination of the probiotic treatment with antibiotics proved to be highly effective. Other studies have shown that probiotic treatment alone had a beneficial effect, yet such an effect often required 3-6 months to be felt (see also J. JAMA, 1996, vol. 275, No 11, U.S. Pat. No. 5,433,826, 1995, U.S. Pat. No. 5,589,168, 1996 and others).

Recent studies have been directed towards investigation of the effects of various types of probiotic bacteria, either alone or in combination; improvement of the survival rate of probiotic bacteria and methods of enabling long-term preservation; biomass accumulation, and the use of probiotic bacteria in prophylaxis and treatment of humans and animals.

Approximately 400 different kinds of bacteria and bacteroids are known to exist in the digestive tract of humans and other mammals, which may provide about 30-40% of excrement volume. The characteristics and functions of only about 15 of these known types have been studied in any detail.

Each of these types of bacteria occupies its own ecological niche in the digestive tract, each having particular conditions for optimal survival and multiplication rate.

Pathogenic bacteria, which may cause various diseases or disorders, also occupy their own particular environmental niches or habitats. Competition between pathogenic and probiotic bacteria may occur under various conditions, but maximal competitive effect occurs when the conditions for optimal survival and multiplication rate of both pathogenic and probiotic bacteria are similar. Under such conditions, survival depends upon more stringent competition for nutrients or growth factors, a well as upon synergistic nutrient utilization and competition for receptor sites. Factors such as production of antimicrobial substances, intensity of multiplication, and creation of restrictive environment, including induction of immunological processes and stimulation of epithelial cell turnover also have great significance under such conditions.

Probiotic compositions have been developed using cultures of non-pathogenic E. coli with other non-pathogenic bacteria (U.S. Pat. No. 5,340,577; U.S. Pat. No. 5,443,826; U.S. Pat. No. 5,478,557; U.S. Pat. No. 5,604,127).

Lactose-positive non-pathogenic E. coli strain having high antagonistic activity have been produced as freeze-dried preparations in Germany and Russia (e.g. use of freeze-dried preparation Colibacterin siccum of E. coli M17, described in Vidal Handbook: Pharmaceutical preparations in Russia, Astra Pharm Service, 1997, Moscow).

In recent years, there has been a move towards developing probiotic preparations comprising a large number (up to 30) different kinds of bacteria and bacteroids (for example, U.S. Pat. No. 5,443,826; U.S. Pat. No. 5,478,557).

However, the advantages of using such a large variety of different bacteria over a preparation including only one or two types have not been proved. In fact, some evidence has been provided that decreased efficiency may occur with a large number of varieties. Behling et al. studied 25 lactose-positive E. coli, and found that two of the types (E. coli 125A and E. coli 128) had inhibitory effects against S. enterids infection in chicks. However, the effect of the 125A strain was significantly greater than that of the 128, and a combination of the two strains produced an effect less than that obtained with the 125A alone.

Studies showed no protection against Salmonella using a mixture of 295 cecal isolates (Goren et al., 1984), whereas protection was provided using a mixture of only 4 isolates (CA patent no. 1,151,066).

Hence, it appears that there is no clear relationship between the number of bacterial strains used and the level of protection obtained. This is due largely to the various interactions between different strains, which may result in synergistic or antagonistic effects.

Studies have been carried out using Lactobacteria, which are dried and incorporated into tiny capsules (U.S. Pat. Nos. 5,501,857; 5,614209; and 5,635,202). The authors claim that such a microencapsulated preparation has greater stability than conventional forms during passage through the stomach.

Studies in preservation of living bacteria have largely been directed towards freeze-dried preparations, with regard to improved production methods and technical solutions for simplifying their application (U.S. Pat. Nos. 5,139,792 and 5,401,501).

Very few prior art patents have dealt with methods of preservation of bacteria in suspension i.e. in the liquid form in a ready-to-use state. U.S. Pat. No. 4,999,301 teaches a method for preservation of Lactobacillus plantrum or Bacillus subtilis microorganisms in a concentrated medium containing between 10-30% nutrient solids for a period of two months. However, this really describes a multi-stage growth cycle, necessitating multiple additional manipulations. At least 0.5% of the bacteria remain viable.

U.S. Pat. No. 4,518,696 teaches a liquid suspension of Lactobacili using an oil-cell mixture with sunflower seed oil. However, the viable cells are dried prior to combining with the oil, and are further characterized by having low internal concentrations of water.

None of the background art patents teaches or suggests a liquid probiotic medium in which the bacterial cells maintain a high level of biological activity. Such a medium is clearly needed, for example for such diseases as inflammatory bowel disease.

Inflammatory bowel disease, or IBD, is a collective term encompassing related, but distinct, chronic inflammatory disorders of the gastrointestinal tract, such as Crohn's disease, ulcerative colitis (UC), indeterminate colitis, microscopic colitis and collagenous colitis, with Crohn's disease and ulcerative colitis being the most common diseases. Another chronic disorder of the gastrointestinal tract is irritable bowel syndrome (IBS).

For most patients, IBD and IBS is a chronic condition with symptoms lasting for months to years. It is most common in young adults, but can occur at any age. It is found worldwide, but it is most common in industrialized countries such as the United States, England, and northern Europe. For example, IBD affects an estimated two million people in the United States alone.

The exact causes of IBD and IBS are not yet understood. Common hypotheses include, for example, disorders in the immune system and actions of pro-inflammatory cytolines and selective activation of lymphocyte subsets, which perpetuate unrestrained activation of an inflammatory response in the intestine. Metabolites generated by pathogenic and potentially pathogenic bacteria may cause disorders in the immune system. Hence, these bacteria may be implicated in disturbances of this nature, related to disturbances in the microbiological balance in the intestine. Such disturbances may themselves be a cause, or alternatively (or in combination), it is believed that this disturbance may in turn lead to auto-immune reactions and/or other reactions of the immune system. For example, it was recently shown that in patients suffering from IBS, up to 70% of such patients have bacterial overgrowth in the intestinal system; treatment of this overgrowth led to a reduction or even cessation of symptoms in many patients with IBS (from research by Dr. Mark Pimentel at Cedars-Sinai Medical Center in California).

IBD and IBS have no cure. Patients afflicted with IBD or EBS are generally treated currently with therapies that are directed at reducing the inflammatory processes, and at reducing the effects of the inflammatory processes on the patients. The presently known medical treatment of IBD is intended to decrease the number, frequency and severity of acute exacerbations of inflammatory bowel disease and to preventing secondary complications, but at best, the results are disappointing.

The presently known methods for treating EBD or IBS may fail to provide a solution for at least some IBD or IBS sufferers as these methods (i) fail to provide a substantial cure for IBD, but rather provide treatment of the symptoms; and (ii) include either drug therapy that is accompanied by severe adverse side effects or invasive surgical treatments, both affecting the sufferer's quality of life.

### SUMMARY OF THE INVENTION

The background art does not teach or suggest a stable, effective liquid probiotic composition. The background art also does not teach or suggest such a composition for treatment of various intestinal disorders, including but not limited to, microbial infection, irritable bowel syndrome (IBS) and inflammatory bowel disease (EBD).

The present invention overcomes this deficiency of the background art by providing a method for preparing a liquid probiotic composition, comprising bacteria having at least a basal biologic activity, wherein said bacteria have been selected according to at least one selection pressure, and wherein the composition is substantially free from substances suitable for bacterial growth but not similarly suitable for mammals, and particularly not suitable for human beings, hereinafter defined as “non-suitable substances”. For example peptone and buffering salts, particularly phosphates, may not be harmful in small doses, but they are not specifically suitable for human beings.

Optionally, the selection pressure may comprise at least one of temperature, time (stability when stored for a period of time), and osmotic pressure. The present invention also provides a method for preparing a liquid probiotic composition, comprising: selecting bacteria according to a selection pressure; and maintaining said bacteria having at least a basal biologic activity for a period of time in storage.

The present invention also provides a method for treating a subject, comprising administering the liquid probiotic composition to the subject in need of treatment thereof. The liquid formulation is therapeutically active immediately following oral administration, as no biomass generation in the gut is required.

Preferably, the method is for treating a gastrointestinal disease or disorder for which treatment is desired or required, which may optionally and more preferably comprise a microbial infection, such as a bacterial infection, and/or IBD and/or IBS. The present invention is also useful for treatment of AAD (antibiotic associated diarrhea), as well as any form of acute diarrhea, for example caused by microbes (including but not limited to, enterotoxigenic E. coli, Salmonella, Proteus, Pseudomonas, Clostridium, Staphylococcus, Shigella flexneri and others), or by undetected pathogens; the syndrome of traveler's diarrhea; acute diarrhea in a hospital setting; as well as for treatment of the symptoms of diarrhea-associated IBS (Irritable Bowel Syndrome) whether mucous or inflammatory, and of diarrhea caused by radiation or chemotherapy.

The present invention is also useful for treatment of the various disease states related to the presence of “abnormal” or an “abnormal” distribution of microflora in the gastrointestinal tract; IBD (inflammatory bowel disease) whether mucous or inflammatory, spastic colon, mucous colitis, antibiotic-associated colitis, idiopathic or simple constipation, and chronic gastrointestinal infections with specific microorganisms such as Clostridium difficile, Campylobacter jejuni/coli etc. and Candida; and chronic diarrhea due to disturbances of the digestive tract microbe balance caused by antibiotics, radiation therapy or chemotherapy, intestinal infection, digestive tract surgery, immunodeficiency, the effects of an unfavorable ecological situation, including higher radiation and age changes.

According to other preferred embodiments of the present invention, the composition and method are optionally useful for treating food intoxication, dyspeptic symptoms or episodes of acute diarrhea, or diarrhea caused by undetected pathogens or unknown etiology. The present invention is also optionally useful for treating diseases and disorders of the digestive tract caused or maintained by disturbance of the microbial balance of the intestinal microflora, and/or by a bacterial overgrowth in the small intestine. The present invention is also optionally useful for preventing or decreasing a level of disturbance microbial balance of the digestive tract microflora resulting from antibiotic therapy, radiotherapy or chemotherapy, diseases or disorders of the digestive tract, including digestive tract surgery.

According to yet other preferred embodiments of the present invention, the composition and method are optionally useful for preventing or treating disturbances in microbial balance of the digestive tract microflora resulting from diseases outside of the digestive tract, certain dietary and environmental factors. The present invention is also useful for improving or normalizing the physiological activity of the gastrointestinal tract in elderly and in the compromised patients.

In a preferred embodiment of the present invention, there is provided a liquid probiotic composition, comprising bacteria having at least a basal biologic activity, in which the bacteria have been selected according to at least one selection pressure, selected from temperature, time stability (stability in storage) and osmotic pressure, and wherein the composition is substantially free from substances suitable for bacterial growth, including bacterial peptones, salts, and also lacking inhibition factors produced by the cells themselves during growth phase. Preferably, the composition comprises an autolysate prepared from the bacteria themselves during an earlier preparation stage, which provides the necessary nutrients for being suitable for maintaining the bacteria in a living state with minimal biological activity, but which are not harmful to lower mammals or humans. Also preferably, the pH of the composition is adjusted to be suitable for maintaining the viability of the bacteria, more preferably from about pH 6 to about pH 7.

Hence, according to one aspect of the present invention there is provided a method of treating an inflammatory bowel disease/irritable bowel syndrome (IBD or IBS, and others) in a subject in need thereof. The method comprising orally administering to the subject a therapeutically effective amount of a probiotic Escherichia coli strain in a liquid formulation. The therapeutically effective amount preferably ranges between about 10 and about 10¹² viable bacteria per administration, ranging from 1 to 10, preferably about 2-4 administrations per day.

According to another aspect of the present invention there is provided a probiotical pharmaceutical composition comprising, as an active ingredient, a probiotic Escherichia coli strain in a liquid formulation.

According to a further aspect of the present invention, there is provided method of treatment for microbial infection, the method comprising orally administering to the subject a therapeutically effective amount of a probiotic strain in a liquid formulation, preferably an Escherichia coli strain.

The table below shows suggested doses of the composition according to the present invention for treatment of various diseases and disorders, and is meant for illustrative purposes only, without wishing to be limiting in any way.

Table of Exemplary Diseases/Disorders and
Suggested Dosing Regimens

| Disease/Disorder | Suggested Doses |
|---|---|
| 1. Diarrhea | |
| Bacterial (Salmonella, Shigella, | 1-3 tablespoons every 3-4 hours |
| Staphylococci, E. coli, Pathogenic | until diarrhea is discontinued |
| serotypes, Klebsiella etc) | or the rate decreases; after which |
| | 1 tablespoon 3 times |
| | per day for 7-10 days |
| Diarrhea associated with antibiotics | 1 tablespoon 3 times per day |
| Traveler's diarrhea | 1-3 tablespoons every 3-4 hours |
| Occasions of acute diarrhea of unknown | 1-3 tablespoons every 3-4 hours |
| etiology | |
| Diarrhea after intestinal surgery or after | 1 tablespoon 2-3 times per day |
| removal of gall bladder | |
| Associated with diabetes | 1 tablespoon 3-4 times per day |
| | for 3-4 months |
| After exposure to radiation and | 1 tablespoon 3 times per day |
| chemotherapy | |
| Age-related | 1 tablespoon 3 times per day for |
| | 3-4 months |
| Viral | 1 tablespoon 3 times per day |
| Parasite related | Preferably as a supplemental |
| | treatment, 1 tablespoon |
| | 3 times per day |
| 2. Constipation | |
| Age related | 1 teaspoon 3 times per day |
| After chemotherapy | 1 tablespoon 3 times per day |
| Associated with diabetes | 1 teaspoon 3 times per day |
| 3. Irritable intestinal syndrome | 1 tablespoon 3 times per day for |
| | 3-4 months |
| 4. pathology (abnormality) in intestinal | 1 tablespoon 3 times per day for |
| micro-ecologic balance (dysbacteriosis, | 3-4 months |
| including candidosis accompanied by | |
| discomfort, excessive flatulence and | |
| periodic pains in the stomach, belching, | |
| bad breath, symptoms indicating | |
| deficiency of vitamins B12, B1, B2, and | |
| so forth | |

The present invention is also useful for improvement or normalizing of the immune system in subjects suffering from an immune system disorder including disorders as side effect caused by therapy of other diseases, as well as being useful for treating domestic animals.

According to still further features in the described preferred embodiments the probiotic non-pathogenic lactose-positive strain, such as the Escherichia coli strain M-17, alone or optionally with one or more E. coli strains and/or other bacterial strains.

According to still further features in the described preferred embodiments the liquid formulation comprises between about 10⁶ and about 10¹² CFU per nil of the probiotic Escherichia coli strain, more preferably from about 10⁷ and about 10¹⁰ CFU per ml of the probiotic Escherichia coli strain.

The present invention successfully addresses the shortcomings of the presently known configurations by providing a method and a probiotic pharmaceutical composition for treating bacterial infections, inflammatory bowel disease/irritable bowel syndrome (IBD or EBS, or others) with a probiotic E. coli strain. Such probiotic treatment is highly advantageous as is compared with the present methods of treating such diseases or disorders as described above, or other diseases or disorders as it is efficacious, safe, non-invasive and side effect-free.

A feature of the present invention is that the E. coli or other bacteria are preserved in biologically active form.

An advantage of the present invention is that the probiotic action of the bacteria commences immediately upon reaching the gastrointestinal tract.

A further advantage of the present invention is that the preparation may be stored for long periods of time without significant loss of bacterial viability.

An additional advantage of the present invention is that the preparation composition of the present invention shows higher stability than dried preparations during passage through the stomach.

An additional advantage of the present invention is that the preparation shows a much more increased effect with larger doses when compared with known probiotic preparations.

Probiotic compositions which are known in the art are clearly inferior, because of the manner in which they have been prepared and stored; for example, as noted above, many such compositions rely upon freeze-drying, which results in a severely decreased level of biological activity.

The present invention also has the advantage that the wide spectrum of efficacy of the liquid probiotic composition enables intestinal infections to allows to be treated effectively without first identifying the pathogen and defining its sensitiveness to antibacterial preparations.

### BRIEF DESCRIPTION OF THE DRAWING

The invention is herein described, by way of example only, with reference to the accompanying drawing:

FIG. 1 shows a comparison of the rate of growth of bacteria taken from the liquid composition according to the present invention with the rate of growth of bacteria taken from a freeze-dried composition; the former clearly shows a superior rate of growth as compared to the latter.

### DETAILED DESCRIPTION

The present invention is of a method for preparing a composition that comprises a non-pathogenic probiotic microorganism, and the composition and the use thereof in the treatment of microbial infections of the gastrointestinal tract, as well as IBS, IBD, antibiotic associated diarrhea (AAD) and any other type of diarrhea or syndrome.

The present invention comprises use of a liquid composition containing probiotic bacteria. Bacterial cells are initially selected by application of selection pressure factors, in order to select those cells which remain viable upon being subjected to conditions unfavorable to metabolism. These selection pressure factors may optionally and preferably include at least one of time stability (stability in storage), temperature, and osmotic pressure conditions. Hence, bacteria having maximum survival ability are selected.

Temperature selection conditions may optionally and preferably comprise subjecting the cells to temperatures which exceed the optimum range for active vital cell metabolism, preferably to temperatures of 40° C. for a period of between 4 and 5 days.

Preferably, cells may be selected by subjecting to temperatures which are below the optimum temperature range for active vital cell metabolism, preferably temperatures of between 2 and 15° C. for a period of between 1-12 months, and more preferably, for between 3 and 12 months.

According to the method of the present invention, selected bacteria are preferably used to inoculate a growth medium, for production of a biomass in order to prepare a liquid probiotic formulation containing selected, viable non-pathogenic bacteria, optionally and preferably comprising between about 10⁷ and about 10⁸ colony forming units of the selected probiotic Escherichia coli per ml. The suspension medium is essentially free from growth medium.

The suspension medium may further comprise a complex of substances which can be used for bacterial cells for maintaining their basal biologic activity with minimum expenditure of energy and plastic metabolism, by being supplied with such substances from the cell suspension itself as a result of autolysis under conditions which prevent production of biodegrading components of bacterial cells. Autolysis may optionally be increased by application of mechanical actions and/or through the composition of the environment. For example, autolysis may be induced by provision of an osmotic imbalance between the osmotic pressure inside the bacterial cell and that of the suspension medium. For example, autolysis may be induced by use of a suitable suspension medium having low osmotic pressure, most preferably 0.3-0.4% sodium chloride solution.

Alternatively, autolysis may be induced through changes to the density of the bacterial suspension, for example by causing the density to preferably be from about 10¹¹ to about 10¹² number of bacterial per ml (CFU; it should be noted that these two terms are used interchangeably in the application).

Also alternatively, another method may be used, to prevent the production of biodegradation components of the bacteria. Examples of such a method include but are not limited to ultrasound or other methods for example.

Optionally and preferably, the bacterial suspension is subjected to conditions favoring autolysis for between 3-7 days in order for accumulation of autolysate to occur, then subjected to conditions of osmotic balance between the interior of the cells and a suitable suspension medium, preferably in the range of from about 0.6% to about 0.7%, most preferably about 0.6% sodium chloride solution.

Cellular components, such as nucleic acid components, accumulate under conditions favorable for autolysis, in quantities preferably of up to 90-110 μg/ml, with cell concentrations of 10¹¹-10¹² number of bacteria per ml.

The suspension medium maintains the cells under conditions which are not only viable, but which also maintain the cells in a biologically active condition. The medium also preferably includes necessary ingredients for maintaining bacteria substantially without further growth or multiplication (as described above), and the medium is essentially free of inhibiting agent normally generated by microorganisms during growth.

The formulation is stored under conditions which maintain the bacteria under viable, biologically active conditions at basal biological activity rate, for maximum time periods. These conditions include pH of between about 6.0 and about 7.0, preferably between about 6.5 and about 6.8, and temperatures of from about 2 to about 10° C.

The liquid formulation of the present invention may be used in treatment of humans and of animals. Preferably, doses of between 10 ml and 20 ml of the formulation are administered to a subject between 2 and 4 times a day.

Hereinafter, the term “substantially free” from a particular substance preferably refers to a condition in which the substance is present in a minor or trace amount, more preferably less than about 5% weight per weight.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the term “teating” includes abrogating, substantially inhibiting, slowing or reversing the progression of a disease, substantially ameliorating clinical symptoms of a disease or substantially preventing the appearance of clinical symptoms of a disease.

The term “preventing” refers to barring a subject from acquiring a disorder or disease in the first place.

As used herein, the phrase “inflammatory bowel disease (IBD)” refers to a disorder or disease characterized by inflammatory activity in the GI tract, and may include mucosal forms of IBD. Examples of IBDs that are treatable by the probiotic strains of the invention include, without limitation, Crohn's disease (both distal and proximal), ulcerative colitis, indeterminate colitis, microscopic colitis, collagenous colitis, idiopathic inflammation of the small and/or proximal intestine and IBD-related diarrhea.

The term “administering”, as used herein, refers to a method for bringing the probiotic E. coli strain(s) or other strain(s) into an area or a site in the GI tract that is affected by the disease or disorder.

The term “therapeutically effective amount” refers to that amount of a probiotic E. coli strain or other strain being administered, which will relieve to at least some extent one or more of the symptoms of the disease or disorder being treated.

Hereinafter, the term “subject” refers to the human or lower animal to which the therapeutic agent is administered.

Compositions for oral administration include suspensions or solutions in water or non-aqueous media or liquid-containing capsules. Thickeners, diluents, flavorings, dispersing aids, emulsifiers or binders may be desirable.

Dosing is dependent on the severity of the symptoms and on the responsiveness of the subject to the therapeutic agent. Persons of ordinary skill in the art can easily determine optimum dosages, dosing methodologies and repetition rates.

A therapeutically effective amount, according to the method of the present invention, preferably ranges between about 10⁶ and about 10¹² viable bacteria per administration, more preferably between about 10⁷ and about 10¹⁰ viable bacteria per administration, more preferably between about 10⁸ and about 10¹⁰ viable bacteria per administration and most preferably it is between about 5×10⁹ and about 2×10¹⁰ viable bacteria per administration.

The number of administrations according to the present invention preferably ranges between 1 and 10 administrations per day, more preferably between 1 and 5 administrations per day and most preferably between 2 and 4 administrations per day. The overall amount of viable bacteria that is administered daily preferably ranges between about 10⁹ and about 10¹¹ viable bacteria per day, although it may optionally range between about 10⁶ and about 10¹² viable bacteria per day.

The probiotic strain of the present invention is preferably formulated and administered as a liquid formulation, as is described in detail hereinbelow and is further exemplified in the Examples section that follows.

The formulation of the probiotic strains of the present invention in a liquid formulation is highly advantageous. Being under biologically active conditions, the formulation serves also as a supportive medium for living bacteria, as opposed to lyophilized formulations, such as the commercial M17 preparation, where the bacteria are under anabiotic conditions. As a result, the liquid formulation of the invention, for example, is therapeutically active immediately following oral administration, as no biomass generation in the gut is required.

The liquid formulation of the probiotic E. coli strain, according to the present invention, typically comprises a suspension of the bacteria in an aqueous solution. The aqueous solution is typically mainly comprised of distilled water, preferably including autolysate from bacteria, salt in an isotonic amount and can further comprise other ingredients, as is further detailed hereinbelow. Preferably, the solution is adjusted for pH which is favorable for maintaining viability. Preferably, the solution also comprises a nitrogen source, but more preferably in a relatively small amount, most preferably less than about 0.3%, and even more preferably less than about 0.03%. It should be noted that unless stated otherwise, all percents herein are given as weight per volume.

The liquid formulation of the probiotic E. coli strain, according to the present invention, typically comprises between about 10⁵ and about 10¹² CFU (colony forming units) of the probiotic Escherichia coli strain, per ml. Preferably, the liquid formulation comprises between about 10⁶ and about 10¹⁰ CFU per ml, more preferably between about 10⁷ and about 10⁸ CFU per ml.

According to a preferred embodiment of the present invention, between 10 ml and 20 ml per day of the liquid formulation is administered to a subject, between 2 and 4 times a day.

The liquid formulation used in context of the present invention is orally administered and as such, it preferably further comprises one or more flavoring agent(s), and/or one or more plant extracts.

Non-pathogenic lactose-positive E. coli, such as strain M17, strain Nisle and other strains comprise the main group of healthy aerobic microflora in the intestine of humans and animals, providing microbiological balance and playing an important role in alimentation and immunity.

This strain of bacteria belongs to the same phylogenetic group as the majority of intestinal pathogens responsible for causing diarrhea, therefore their survival conditions are largely similar, resulting in a high level of competitive exclusion between the strains. This competitive effect includes production of antimicrobial substances during growth of probiotic bacteria, competition for nutrients and growth factors, synergistic nutrient utilization, and competition for receptor sites.

The speed of multiplication, which is a major factor in competitive antagonism, is higher with the bacterial strain of the present invention, than with, for example, Lactobacillus or B. Bifidus, and is at least equivalent to that of many intestinal pathogens. Furthermore, the bacterial strain of the present invention is much less selective than strains such as Lactobacillus or B. Bifidus with regard to nutritional requirements.

Currently available probiotic preparations use dried bacteria, such that the bacteria remain alive, but in anabiotic condition. Upon administration of such a preparation, a lag period occurs before biological activity is recovered. Since the contents of the intestine are rapidly expelled in the event of diarrhea, only a small percentage of administered dried bacterial preparation is retained in the colon to multiply and acquire biological activity.

The liquid probiotic composition of the present invention enables preservation of the bacteria in biologically active form, such that the probiotic action of the bacteria commences immediately upon reaching the gastrointestinal tract, with no adaptation time required. The time taken for bacterial growth to commence is therefore much faster with the liquid composition of the present invention than with preparations using freeze-dried bacteria.

The antagonistic effect of the probiotic composition of the present invention on bacterial pathogens was found to be considerably higher than that of probiotic bacteria from freeze-dried preparations. It should be noted that by “antagonistic”, it is meant the ability of a particular bacterial strain to antagonize growth of other bacteria or other micro-organisms.

It is known that the action of gastric juice, largely comprising hydrochloric acid, causes death of many bacteria. Bacteria in dried form are weaker than those contained in liquid medium, and are therefore more susceptible to the effects of gastric juice. The bacteria contained in the liquid composition of the present invention are therefore more stable upon passage through the stomach than those in freeze-dried preparations. Bacteria entering the colon begin to multiply and exert their antagonistic properties. However, the site of primary action for the majority of intestinal pathogens is not the colon but the upper part of the gastrointestinal tract. Known probiotic preparations do not enable delivery of a competitive concentration of live bacteria to the upper portions of the intestine, and are therefore practically ineffective in eliminating acute bacterial diarrhea and conditions caused by disturbance of the micro-ecological balance in upper sections of the intestine.

Using conventional probiotic preparation production methods, increasing the bacterial quantity of the preparation is problematic. In such methods, the bacteria are dried together with the culture medium and various stabilizing agents are added to increase bacterial stability. Increasing the quantity of bacteria administered therefore results in increase in the consumption level of the other components, which can lead to serious side-effects.

In contrast, the composition of the present invention comprises liquid suspension of biologically pure bacteria, such that the number of bacteria administered per day may be varied, from several tens of millions, up to about 200 billion bacteria, in a volume preferably up to about 150 ml, and at a concentration: as previously stated. This enables a working competitive concentration of bacteria to be provided which begin with the upper sections of digestive tract, i.e. the site at which the majority of intestinal pathogens act. The target site determines the concentration which is required to treat the disease or disorder.

The effectiveness of the liquid probiotic composition of the present invention is also increased due to the fact that it may be administered in accordance with the dosing frequency determined as providing maximum dose dependant efficacy according to the disease or disorder to be treated. For example, in treating acute diarrhea, the liquid probiotic composition of the present invention can be administered in a quantity 10-100 times higher than the effective quantity used for treatment of constipation.

In preparation of the liquid probiotic composition of the present invention, the E. coli bacterial cells (or other bacterial cells) having the highest antagonistic activity and the most persistent bacterial cells under storage for long periods of time, preferably up to about 12 months, are more preferably first selected from lactose-positive non-pathogenic E. coli species having beneficial probiotic properties.

E. coli cells or other bacteria for use in the probiotic composition of the present invention are selected by exerting selection pressure on the cells such that only selected cells remain viable. Application of selection pressure may be achieved by use of time pressure (stability over time), such that cells having long-term survival ability are selected; application of osmotic pressure; decrease of basal metabolism; or increase in temperature. Temperature selection optionally and preferably comprises subjecting the cells to temperature of 40° C. for at least 4 days, and/or to higher temperatures for a shorter period of time. By these means, only cells having high survival abilities are selected from the initial culture.

The selected bacterial cells were used for inoculation of growth medium, as described in greater detail below, with reference to Examples 6 and 7. The nutrient composition of the present invention may comprise various factors such as described with reference to the examples, for example from yeast extract and/or yeast autolysate. The nutrient composition of the growth medium of the present invention may optionally include growth factors and provides a considerable increase in the accumulated bacterial biomass relative to that obtained with conventional growth media because of the addition of such growth factors, for example from yeast extract, which results in an economic benefit. The yeast extract is preferably present in an amount from about 5 gram per liter to about 25 gram per liter, and more preferably from about 15 gram per liter to about 20 gram per liter.

Other sources of the nutrient composition are possible, but preferably include all of the necessary nutrients, growth factors etc as are known in the background art, such as described for example in “Manual of Methods for General Bacteriology”, P. Gerhardt ed., American Society for Microbiology, Washington, D.C., USA, 1981.

The method of the present invention provides biologically pure bacteria, free from culture medium, with its associated side-effects upon administration of large quantities, and from the inhibiting agents normally produced by the microorganism during growth, which delay commencement of growth and activity of bacteria.

It is known that osmotic pressure inside cells of Gram-negative bacteria, particularly E. coli, may reach up to 15 atmospheres in the log phase of growth, and 2-3 atmospheres in the stationary phase of growth. In a preferred embodiment of the method of the present invention, a suspension medium having low osmotic pressure, preferably below 1 atmosphere, more preferably from about 0.3 to about 0.4 atmospheres, is used. Osmotic imbalance and high bacterial density during the first preparation stage of the liquid probiotic composition of the present invention creates conditions for autolysis of the weakest and smallest stable bacterial cells in the log phase. These lysed cells provide an accumulation of cellular components from bacteria in the suspension medium, which provide nutritional requirements of remaining cells. Using this procedure, cell concentrations of from 10¹¹ to about 10¹² bacteria per ml (CFU) were obtained, although again cell concentrations may optionally be present in a broader range.

As shown in Example 1, the pH of the suspension medium of the present invention for maximum cell stability is optionally and preferably in the range of from about 6.0 to about 7.0. More preferably, the pH of the suspension medium is about 6.5.

As shown in Example 2, the bacterial cells of the present invention are optionally and preferably stored at temperatures in the range of from about 2 to about 20° C. More preferably, the storage temperature is in the range of from about 20 to about 10° C. Most preferably, the storage temperature is in the range of from about 2 to about 4° C.

Under the conditions favorable to cell stability (for example, suitable pH, cell concentration of 10⁷-10⁸ bacteria per ml, complex of substances used by bacterial cells for maintaining their structure with minimum expenditure of energy and plastic metabolism etc. (autolysate)), the liquid probiotic composition of the present invention creates a combination of factors which preserve the bacteria not only in a viable condition, but also in an immediately active biological form, for at least 12 months. It should be noted that the concentration of bacteria for this stage may optionally range from about 10⁶ to about 10¹² bacteria per ml. This complex of substances preferably includes nucleic acids, nucleic acid components, bacterial lipopolysaccharides, peptidoglycans, phospholipids and many other desirable substances.

The probiotic composition of the present invention may be used in treatment of humans and of animals.

### EXAMPLES

The formulation, preparation and use of the probiotic composition of the present invention is illustrated with reference to the following non-limiting examples.

### Example 1

### Process for the Preparation of the Liquid Probiotic Composition

The selected bacteria were first prepared for growth to form the biomass in the form of concentrates ranging from 10¹¹-10¹² CFU per ml in 0.3%-0.4% NaCl solution, to produce the autolysate.

For preparing the liquid probiotic composition, cell concentrates were diluted in 0.6%-0.8% NaCl solution at a cell concentration of 10⁷ cells/ml (although again optionally the concentration of bacteria may range from about 10⁶ to about 10¹² bacteria per ml). The liquid probiotic composition was adjusted to a pH favorable to cell survival. The preferred pH was from about 6.5 up to 6.8. For improvement of the taste, one or more plant extracts, flavoring agents and/or other additives may be added, which do not decrease viability of bacteria preserved for long periods of time. A description is provided below of an exemplary method for preparing the plant extracts in Example 15.

The liquid probiotic composition may be preserved under refrigerated conditions for at least 12 months without significant loss of biological properties.

### Example 2

Viability of bacteria E. coli M-17* (CFU/ml) depending on pH of the suspension medium (0.7% Sodium Chloride solution with the autolysate, which together provides an osmotically balanced solution) at temperature +2±8° C.

| Time of | | | | | | | | |
|---|---|---|---|---|---|---|---|---|
| storage | pH of suspension medium | | | | | | | |
| (months) | 5 | 5.5 | 6.0 | 6.5 | 7.0 | 7.5 | 8.0 | 8.5 |

| | | | | | | | | |
|---|---|---|---|---|---|---|---|---|
| 0 | 10⁸ | 10⁸ | 10⁸ | 10⁸ | 10⁸ | 10⁸ | 10⁸ | 10⁸ |
| 1 | 10⁵ | 10⁵ | 10⁷ | 10⁸ | 10⁸ | 10⁶ | 10⁵ | 10² |
| 2 | 10³ | 10⁴ | 10⁶ | 10⁷ | 10⁷ | 10⁴ | 10⁴ | <10¹ |
| 3 | <10¹ | 10² | 10⁶ | 10⁶ | 10⁶ | 10⁴ | 10³ | <10¹ |
| 6 | <10¹ | <10¹ | 10⁴ | 10⁵ | 10⁵ | 10³ | <10¹ | |
| 9 | <10¹ | <10¹ | 10³ | 10⁴ | 10⁴ | <10¹ | <10¹ | |
| 12 | <10¹ | <10¹ | 10² | 10³ | 10² | <10¹ | <10¹ | |

As shown above, the number of viable cells greatly decreases within one month upon storage in suspension medium having pH of 8.5. A significant decrease is seen within 2 months of storage at pH of less than 5.5 or greater than 7.5. By the end of 12 months, significant numbers of viable cells remain only in those media having pH of between 6.0 and 7.0.

### Example 3

Viability of E. coli M-17 bacterial cells after selective sampling according to the present invention (1) and those isolated from commercial freeze-dried preparation (2) in suspension, depending on storage temperature. 0.7% sodium chloride solution was used as suspension medium. Suspension pH=6.7.

| Time of | Log No Bacteria per ml in temperature intervals | | | | | | | |
|---|---|---|---|---|---|---|---|---|
| exposure | 2° C.-4° C. | | 8° C.-10° C. | | 18° C.-20° C. | | 25° C.-30° C. | |
| (months) | 1 | 2 | 1 | 2 | 1 | 2 | 1 | 2 |
| 0 | 10⁸ | 10⁸ | 10⁸ | 10⁸ | 10⁸ | 10⁸ | 10⁸ | 10⁸ |
| 1 | 10⁸ | 10⁶ | 10⁸ | 10⁶ | 10⁸ | 10⁵ | 10⁷ | 10³ |
| 2 | 10⁸ | 10⁶ | 10⁸ | 10⁵ | 10⁸ | 10⁴ | 10⁶ | 10² |
| 3 | 10⁸ | 10⁵ | 10⁸ | 10⁵ | 10⁷ | 10³ | 10⁵ | <10¹ |
| 6 | 10⁸ | 10⁵ | 10⁸ | 10⁴ | 10⁶ | <10¹ | 10³ | <10¹ |
| 9 | 10⁸ | 10⁴ | 10⁷ | 10⁴ | 10⁶ | <10¹ | 10² | <10¹ |
| 12 | 10⁷ | 10² | 10⁷ | 10³ | 10⁵ | <10¹ | <10¹ | <10¹ |

As shown above, the number of viable bacteria in suspension decreases very little over a 12 month period of storage at temperatures of between 2 and 10° C., while a significant decrease is seen in the same time period with storage at 18-20° C. At temperatures of 25-30° C., almost no viable cells remain after 12 months.

### Example 4

The same quantity of bacteria E. coli M-17 (1 ml 10⁸ CFU per each sample) from freeze-dried live bacteria (commercial preparation Colibacterin) and from liquid probiotic composition (bacteria are present in biologically active form) was introduced into nutrient broth samples (200 ml). The mixture was incubated at 37° C. for 90 minutes.

The rate at which bacterial metabolic activity was reduced, i.e. their adaptation to the conditions of the new environmental medium, was evaluated in relation to the rate of their initial growth. To obtain this value, the quantity of bacteria (C.F.U. per 1 ml) was determined immediately after introducing bacteria into the nutrient broth, and subsequently at intervals of 30 minutes during the incubation process.

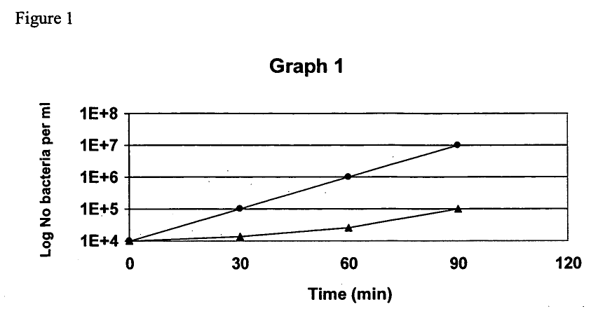

The growth of bacteria using cells taken from the liquid probiotic composition (∘) was considerably faster than the bacterial growth rate (Δ) with cells from the freeze-dried probiotic, as shown in FIG. 1.

### Example 5

10-20 hour cultures of S. flexneri and S. sonnei were diluted in saline to a concentration of 10⁵ CFU/ml. These were then seeded (1 ml) either alone or in combination with cultures of E. coli M17 (diluted in saline to a concentration of 10¹² CFU/ml) from freeze dried probiotic (Colibacterin) or from liquid probiotic (Bio-Co) in test tubes containing Nutrient Broth (5 ml). The tubes were incubated for 24 hours at 37° C. The number of colony-forming units (CFU) of the pathogens and of E. coli M17 from both probiotic preparations was determined by plate counts on nutrient agar.

These data are shown in the table below.

Antagonistic activity of probiotic organism E. coli M17
from freeze dried and from liquid probiotic composition
in relation to various strains of Shigella.

| | The culture growth(Shigella/E. coli M17) | | |
|---|---|---|---|
| | from mixed bacterial medium | | |
| | (CFU/ml) | | |
| Probiotic | Shigella flexneri 1a | Shigella flexneri 2a | Shigella sonnei |
| Freeze dried | 7/195 | 8/181 | 13/300 |
| liquid | 2/196 | 0/240 | 0/160 |
| .(Shigella | 60 | 7 | 18 |
| alone as a | | | |
| control) | | | |

### Example 6

Culture medium (solid and liquid), shown as tryptic soy agar (TSA) and tryptic soy broth (TSB) was seeded with the same quantity of bacterial culture E. coli M17. For a reference to the composition and preparation of TSA and TSB, which are well known in the art, see for example “Culture Media for Microbiology”, FEROSA/Scharlau, 1996, which is hereby incorporated by reference as if fully set forth herein. The microorganism was cultivated under aerobic conditions at the optimum temperature (36° C.) for 24 hours (solid culture media) and for 18 hours (liquid culture media). The concentration of bacteria per 1 ml was then determined by plate counts on nutrient agar.

A comparison of accumulation of biomass of non-pathogenic E. coli (E. coli M17) on an optimum growth medium (T.S.A. and T.S.B.) and on a growth medium in accordance with the invention is illustrated in table below.

The culture growth on different culture media

| | Concentration of bacteria |
|---|---|
| Culture media | (Log ivo per 1 ml culture media) |
| Solid culture media of the invention | 10¹⁰-10¹¹ |
| T.S.A. | 10⁸-10⁹ |
| Liquid culture media of the invention | 10¹²-10¹³ |
| T.S.B. | 10¹⁰-10¹¹ |

### Example 7

An analysis of nucleic acid components was carried out on samples of a 10¹¹-10¹² CFU suspension of the bacteria in 0.4% sodium chloride solution after preservation of samples for 3 and 30 days at temperature +2±4° C.

Initially, the samples were filtered through a 0.45μ microbiological filter in order to obtain a cell-free filtrate. The filtrates were checked for presence of nucleic acid components (adenine and uracil). According to the sensitivity of the methods, the limit of detection is 2 μgr/ml. These data are shown in the table below.

| Sample after preservation | | |
|---|---|---|
| for | μgr/ml adenine | μgr/ml uracil |
| 3 days | >0.2 | 7.4 |
| 30 days | >0.2 | 102.1 |

### Example 8

### Production of Bacterial Biomass Using Liquid Culture Media

For bacterial biomass preparation a standard fermentation vessel with aeration can be used. Nutrients necessary for bacterial growth are added in two stages.

In a typical fermentation process, a medium may consist of soy peptone at 10.0 g/l, yeast extract at 18.0 g/l, glucose at 2.5 g/l, sodium chloride at 3.0 g/l, and a combination of disodium phosphate and monopotassium phosphate sufficient to provide a neutral or slightly basic pH (7.2±0.2).

Additional nutrients are automatically supplied into the nutrient medium during the process of bacterial growth.

Additional glucose should be continuously added following the growth of the culture in such way that the glucose concentration in the fermentation broth is kept at the level of 2.5 g/l.

Additional aeration (0.5 vvm.) is performed during the entire period of bacterial growth.

The pH of the fermentation broth may be kept neutral by the continuous addition of 4N NH₄OH.

The broth is incubated at temperatures of from about 32 to about 36° C. until the stationary phase of the growth cycle is reached.

After 16-18 hours, the cells are harvested by centrifugation or ultrafiltration, up to a level at which residual quantities of total nitrogen are not more than 0.3%, and preferably not more than 0.03% for cell concentration of 10⁷-10⁸ microbial cells per ml of suspension, resuspended in saline and re-precipitated.

A 10¹¹-10¹² suspension of the bacteria is prepared in 0.4%-0.6% NaCl solution cooled to 4-8° C. and, stored under refrigerated conditions. It should be noted that the concentration of bacteria for this stage (and/or for administration to the subject) may optionally range from about 10⁶ to about 10¹² bacteria per ml.

### Example 9

### Production of Bacterial Biomass Using a Solid Culture Media

Non-pathogenic E. coli were grown on a solid culture medium, using a composition of nutrients providing maximum accumulation of bacterial biomass according to the present invention.

The composition of the medium is as follows:

| Formula (in g/l) | |
|---|---|
| Soy peptone | 10.0 |
| Yeast extract | 18.0 |
| Dextrose | 2.5 |
| Sodium chloride | 4.0 |
| Agar | 12.0 |

Final pH 7.0 (0.2 approx.)

Prepared medium is poured into corresponding matrices with layer thickness of 5-7 millimeters. After cooling, the culture medium is seeded with bacterial culture E. coli M-17.

Matrices are placed in an incubator and incubated under aerobic conditions at the optimum temperature (34-38° C.) for about 24-28 hours. This procedure yielded 10¹⁰-10¹¹ cells/ml of the culture medium.

After this period, the isolated pure culture should be removed from plates by “Dry method”, in which the bacteria are removed with a tool such as a spatula, without introducing a liquid (or at least substantially quantities of a liquid) to the plates. For this purpose special adjustments for biomass collection have been used.

A 10¹¹-10¹² CFU suspension of the bacteria is prepared in 0.4%-0.6% NaCl solution. The suspension is stored under refrigerated conditions.

### Example 10

### Treatment of Diarrhea

The effect of eliminating episodes of acute diarrhea caused by Salmonella and food intoxications of unknown etiology (including traveler's diarrhea) depending on the quantity of probiotic bacteria administered to a patient per day is shown (dose-dependent efficacy).

A total of 64 patients were treated with different therapeutically effective amounts of the liquid probiotic composition of the present invention. These quantities were in the range of 10-200 billion live bacteria per day, divided into 4-6 doses.

A first group of patients were prescribed the liquid probiotic composition of the invention containing a therapeutically effective amount of 10 million bacteria per day. In 85% of these patients, the symptoms of acute diarrhea were still present after 3 days. From the 4^{th} day, these patients were prescribed the therapeutically active amount of 200 billion bacteria per day. Diarrhea disappeared or the number of defecations considerably decreased in 94% of patients within the first day of administration of this dose.

A second group of patients were administered a therapeutically effective amount of liquid probiotic composition of the present invention comprising 200 billion live bacteria from the first day of observation. Pronounced effect (disappearance of diarrhea or considerable decrease in the number of defecations) was noticed even within the first day of administering the liquid probiotic composition, mainly during the first 12-14 hours.

### Example 11

### Effect of Liquid Probiotic Composition on the Character of Intestinal Microflora Changes in Patients Treated with Anti-Helicobacter Therapy

A group of 104 patients treated with anti-helicobacter therapy was randomly divided into 2 sub-groups.

In addition to standard therapy including treatment with antibiotics, the patients of the 2^{nd} group were administered the liquid probiotic composition of the present invention. After 25 days of treatment, the quantitative composition of bacteria considered to be the main representatives of healthy microflora (aerobic as well as anaerobic) was determined in all the patients.

The results are shown in the Table below.

| | Data in the groups | | |
|---|---|---|---|
| | | subjects undergoing | |
| | healthy subjects | anti-Helicobacter therapy | |
| Microorganism | (as control) | 1 | 2 |
| Total quantity E. | 300-400 | 124 | 318 |
| coli (million/g) | | | |
| Lactose negative | 5 | 17 | 6 |
| E. coli (% of total | | | |
| quantity E. coli) | | | |
| Bifidobacteria | 10⁹ | 10⁷ | 10⁸ |
| (medium log) | | | |
| Lactobacteria | 10⁷ | 10⁶ | 10⁷ |
| (medium log) | | | |

Non-pathogenic E. coli of the type M17 serves as the leading representative of aerobic microflora in the intestine. Upon administration of the liquid probiotic composition of the present invention, the total number of E. coli in the intestine is normalized, and their quality improved (i.e. the level of lactose-positive bacteria is increased, and the level of lactose-negative bacteria and E. coli with low fermentative activity is decreased). Furthermore, the number of bifidobacteria, which are the most important representatives healthy anaerobic intestinal microflora, is increased.

### Example 12

### Preparation of the Probiotic Composition—Exemplary Method

The composition according to the present invention may optionally be prepared according to the following exemplary method. Probiotic E. coli (10⁸-10⁹ cells), optionally from a seed stock, are inoculated into liquid or solid culture medium components using standard microbial fermentation techniques. Growth conditions preferably include continuous aeration, maintenance of neutral pH and supplementation with glucose. This organism has preferably not been genetically engineered in any way, but rather has been isolated from microflora obtained from a normal human gastrointestinal tract.

Manufacturing is optionally and preferably controlled with respect to the following critical control points:
Precautions to be taken receiving and handling cultures
Control procedures to assure appropriate culture conditions
Maintenance of sterility
Control procedures to assure correct levels of probiotic in finished product

Optionally and preferably the seed stock itself may be prepared as follows. One frozen vial of E. coli M-17 strain is removed from storage at −80° C., thawed at room temperature, and then transferred aseptically into a sterile baffled shake flask containing sterilized Tryptic Soy broth (Difco). After 15-20 hour's growth, the culture is examined microscopically and streaked onto a Bacto m Endo Agar LES plates to check for purity.

Reactor Preparation

Each reactor is batched and sterilized with the medium in place. Dextrose is sterilized separately and added to a concentration of 2.5 g/L before culture inoculation.

Reactor Inoculation

The seed culture is aseptically transferred to the bioreactor, and the culture grown under established conditions of temperature, pH, agitation and dissolved oxygen. A glucose feed of 3.5 to 3.9 g/L is started fours hours post inoculation. After 18-22 hour's growth, the culture is examined microscopically and streaked onto a Bacto m Endo Agar LES plates to check for purity. The reactor is then cooled to <10° C. for harvest.

Microfiltration

Bioreactor contents are harvested by concentration using a 0.2 μm pore size tangential flow microfiltration unit. Concentrate is diafiltered with 5 volumes of sterile saline and then placed into sterile bottles for storage at 4-6° C. The sample is examined microscopically and streaked onto a Bacto m Endo Agar LES plate to check for purity and enumerated by plating onto Tryptic Soy agar plates.

### Example 13

### Treatment of IBD-Related Diarrhea Using E. coli

A 23-year-old male, suffering from loose bowel movements with episodes of diarrhea for two years, and having no rectal bleeding or weight loss, was studied. His family history was unremarkable for bowel disease.

Laboratory tests of the patient showed the following: Hemoglobin=17.6 (smoker), ESR=10, Platelets=219, Albumin=4.1, tissue transglutaminase TTG=29.8 (normal <20).

In addition, the patient was found to have lactose intolerance by a positive H2 breath test. However, a diet free of dairy products did not improve his condition.

The patient's elevated TTG value suggested a diagnosis of Celiac disease. An upper GI endoscopy revealed a normal appearing small bowel. A random biopsy from the second part of the duodenum documented the presence of normal small bowel mucosa. A capsule endoscopy study was preformed and revealed inflammatory changes in the proximal small bowel including a few erosions, mucosal hemorrhages, edema and loss of villi.

The patient was treated with a probiotic composition prepared as described in Example 12 at a regimen of one tablespoon twice daily, approximately half an hour before meals. After 2 weeks there was no improvement in the patient's condition. The probiotic treatment of the patient was continued while raising the daily dose to four tablespoons daily. Following this treatment, the patient reported substantial improvement, for the first time in two years. A second capsule endoscopy of the small bowel demonstrated improvement of the inflammatory process of the proximal small bowel.

### Example 14

### Methods of Treatment with the Liquid Probiotic Composition

As noted above, the liquid probiotic composition of the present invention have been shown to be effective treatments for gastrointestinal diseases and conditions, including but not limited to, microbial infection, IBS and IBD. The following example is an illustration only of a method of treating such a gastrointestinal disease or disorder (or condition in need of treatment), and any other suitable condition with the liquid probiotic composition of the present invention, and is not intended to be limiting.

The method includes administering the liquid probiotic medium to a subject to be treated. The liquid probiotic composition is administered in a pharmaceutically effective amount according to an effective dosing methodology, preferably until a predefined endpoint is reached, such as the absence of a symptom of a gastrointestinal disease, disorder or condition and any other suitable condition in the subject, or the prevention of the appearance of such a disease, disorder, condition or symptom in the subject.

### Example 15

### Preparation of the Plant Extract

The biologically active edible extract may be prepared from any suitable fruit, vegetable, leaf, stem, or root of a plant, or from herbs. The plant can be cabbage, garlic, parsley, dill, lemon and etc, or a herb such as mint and so forth.

The plant extract may optionally be prepared by distillation under reduced pressure providing boiling temperature up to 40° C.

For plant extracts preparation, equipment existing in the market can be used, for instance the “Rotovapor” device.

A process for preparing a plant extract for being added to the composition of the present invention preferably includes:

1. Grinding a plant or plant part to give a plant biomass.

It is to be stressed that for plant extract production freshly prepared biomass is to be used. It is preferably stored for no more than 1-2 hours at room temperature, as after crushing fruit, vegetables or other plants: microorganisms begin to develop in biomass, and uncontrollable fermentative and other reactions take place. This considerably lowers the quality of the extracts obtained. In case the prolonged storage is necessary, grinding plant matter should be stored in a refrigerator for no longer than 12 hours.

2. Steam distilling the plant bio mass under reduced pressure.

3. Collecting the volatile fraction obtained from said steam distillation. This fraction may be further diluted with water.

The plant extract can optionally be stored for 12 months under refrigerated conditions without loosing its capacity. This fraction itself constitutes a food/feed additive and may also optionally be prepared by mixing more than one plant extract.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A liquid probiotic composition, comprising bacteria having at least a basal biologic activity and a liquid medium, wherein said bacteria have been selected according to at least one selection pressure, and wherein the composition is substantially free from non-suitable substances.

2. The composition of claim 1, wherein said bacteria comprise at least one strain of E. coli.

3. The composition of claim 2, wherein said bacteria comprise a non-pathogenic lactose-positive strain having antagonistic properties.

4. The composition of claim 2, wherein said bacteria comprise a plurality of strains of Escherichia coli, or at least one strain of E. coli with at least one additional bacterial strain.

5. The composition of claim 1, wherein said selection pressure comprises temperature pressure.

6. The composition of claim 5, wherein said temperature pressure comprises raising a temperature of a medium containing said bacteria.

7. The composition of claim 6 wherein said temperature pressure comprises subjecting said bacteria to temperature of from about 36 to about 50° C., wherein said bacteria are in suspension.

8. The composition of claim 7, wherein said bacteria are subjected to said temperature of about 40° C. for at least 4 days.

9. The composition of claim 5, wherein said temperature pressure comprises lowering a temperature of a medium containing said bacteria.

10. The composition of claim 9, wherein said lowering comprises lowering said temperature to from about 1° C. to about 12° C. for up to 12 months.

11. The composition of claim 10, wherein said temperature is lowered for at least about 3 months.

12. The composition of claim 1, wherein said selection pressure comprises time in storage, wherein said bacteria are stored for at least about one month.

13. The composition of claim 12, wherein said bacteria are stored for up to about 12 months.

14. The composition of claim 1, wherein said selection pressure comprises osmotic pressure.

15. The composition of claim 14, wherein said selection pressure comprises low osmotic pressure.

16. The composition of claim 15, wherein said osmotic pressure comprises a pressure below about 1 atmosphere.

17. The composition of claim 16, wherein said osmotic pressure comprises a pressure from about 0.3 to about 0.4 atmospheres.

18. The composition of claim 1, wherein said liquid medium comprises water, autolysate and an isotonic amount of salt, being characterized in that nitrogen source is present at a level of less than about 0.3%, optionally less than about 0.03%.

19. The composition of claim 7, further comprising a plurality of substances for maintaining bacteria substantially without growth or division.

20. The composition of claim 1, comprising from about 10⁷ to about 10⁸ colony forming units of said bacteria per ml of said liquid.

21. The composition of claim 1, further comprising at least one of a flavoring agent or an extract from a plant.

22. The composition of claim 1, comprising pH of about 6.0 to about 7.0.

23. The composition of claim 14, wherein said pH is about 6.5.

24. The composition of claim 1, comprising the composition in a dosage form suitable for administering from about 10⁶ to about 10¹² viable bacteria per administration.

25. The composition of claim 24, comprising the composition in a dosage form suitable for being administered in from about 1 to about 4 administrations per day.

26. The composition of claim 1, wherein said bacteria are subjected to autolysis before being combined with said liquid medium.

27. The composition of claim 26, wherein said autolysis is induced through a mechanism selected from the group consisting of mechanical agitation, low osmotic pressure and a change to a density of said bacteria in a suspension.

28. The composition of claim 27, wherein said density is from about 10¹¹ to about 10¹² bacteria per ml.

29. The composition of claim 26, wherein said liquid medium comprises an autolysate formed from said autolysis.

30. A method for preparing a liquid probiotic composition, comprising:
growing a plurality of bacteria to form a biomass in a growth media;
selecting bacteria according to a selection pressure; and maintaining said bacteria having at least a basal biologic activity for a period of time in storage.

31. The method of claim 30, wherein said selection pressure comprises temperature pressure.

32. The method of claim 31 wherein said temperature pressure comprises subjecting said bacteria to temperature of from about 36 to about 50° C., wherein said composition is in suspension.

33. The method of claim 32, wherein said bacteria are subjected to said temperature of about 40° C. for at least 4 days.

34. The method of claim 31, wherein said temperature pressure comprises lowering a temperature of a medium containing said bacteria.

35. The method of claim 34, wherein said lowering comprises lowering said temperature to from about 1° C. to about 12° C. for up to 12 months.

36. The method of claim 35, wherein said temperature is lowered for at least about 3 months.

37. The method of claim 30, wherein said selection pressure comprises time in storage, wherein said bacteria are stored for at least about one month.

38. The method of claim 37, wherein said bacteria are stored for up to about 12 months.

39. The method of claim 30, wherein said selection pressure comprises osmotic pressure.

40. The method of claim 39, wherein said selection pressure comprises low osmotic pressure.

41. The method of claim 40, wherein said osmotic pressure comprises a pressure below about 1 atmosphere.

42. The method of claim 41, wherein said osmotic pressure comprises a pressure from about 0.3 to about 0.4 atmospheres.

43. The method of claim 30, further comprising:
Separating said biomass from said growth media and from inhibitor factors; and
Subjecting said bacteria to autolysis.

44. The method of claim 43, wherein said autolysis is induced through a mechanism selected from the group consisting of mechanical agitation, osmotic change, low osmotic pressure and a change to a density of said bacteria in a suspension.

45. The method of claim 44, wherein said density is from about 10¹¹ to about 10¹² bacteria per ml.

46. The method of claim 45, further comprising storing said bacteria with a liquid medium, wherein said liquid medium comprises an autolysate formed from said autolysis.

47. The method of claim 46, wherein said storing comprises adjusting a concentration of said bacteria in said liquid medium to from about 10⁵ to about 10¹² CFU.

48. The method of claim 47, wherein said concentration is from about to about 10⁸ CFU.

49. The method of claim 30, wherein said growth medium comprises a solid or a liquid growth medium.

50. The method of claim 30, wherein said growth medium comprises yeast extract.

51. The method of claim 50, wherein said yeast extract is present in an amount of from about 5 to about 25 gram per liter.

52. The method of claim 51, wherein said yeast extract is present in an amount of from about 15 to about 20 gram per liter.

53. The method of claim 30, further comprising:
Preparing a plant extract by grinding plant material and steam distilling the ground material; and
Adding said plant extract to said composition.

54. The method of claim 53, wherein said steam distilling is performed under reduced pressure.

55. A method for treating a subject, comprising administering to the subject in need of treatment thereof, a liquid probiotic composition, comprising bacteria having at least a basal biologic activity, wherein said bacteria have been selected according to at least one selection pressure.

56. The method of claim 55, for use in treatment of a gastrointestinal disease or disorder.

57. The method of claim 56, wherein said gastrointestinal disease or disorder comprises a microbial infection.

58. The method of claim 56, wherein said gastrointestinal disease or disorder comprises inflammatory bowel disease.

59. The method of claim 58, wherein said inflammatory bowel disease is selected from the group consisting of Crohn's disease, ulcerative colitis, indeterminate colitis, microscopic colitis, collageneous colitis, idiopathic inflammation of the small intestine, and inflammatory bowel disease-related diarrhea.

60. The method of claim 56, wherein said gastrointestinal disease or disorder comprises a gastro-enteric infection caused by an enteric pathogen selected from the group consisting of Salmonella, Shigella, enterotoxigenic E. coli, and C. difficile.

61. The method of claim 56, wherein said gastrointestinal disease or disorder comprises a food intoxication, dyspeptic symptoms or episodes of acute diarrhea, or diarrhea caused by undetected pathogens or unknown etiology.

62. The method of claim 61, wherein said diarrhea comprises traveler's diarrhea and diarrhea in a hospital setting.

63. The method of claim 55, comprising treating diseases and disorders of the digestive tract caused or maintained by disturbance of the microbial balance of the intestinal microflora, and/or by a bacterial overgrowth in the small intestine.

64. The method of claim 55, comprising preventing or decreasing a level of disturbance microbial balance of the digestive tract microflora resulting from antibiotic therapy, radiotherapy or chemotherapy, diseases or disorders of the digestive tract, including digestive tract surgery.

65. The method of claim 55, comprising preventing or treating disturbance in microbial balances of the digestive tract microflora resulting from diseases out from digestive tract, certain dietary and environmental factors.

66. The method of claim 55, comprising improving or normalizing the physiological activity of the gastrointestinal tract in elderly and in the compromised patients.

67. The method of claim 55, wherein said composition optionally comprises between about 10⁶ to 10¹² colony forming units per ml of said bacteria, and preferably comprises between about 10⁷ to 10⁸ colony forming units per ml of said bacteria.

68. The method of claim 67, wherein from about 10⁶ to about 10¹² of said bacteria are administered to the subject per day.

69. The method of claim 68, wherein from about 10⁷ to about 10⁹ of said bacteria are administered to the subject per day.

70. The method of claim 69, wherein from about 10⁷ to about 10⁸ of said bacteria are administered to the subject per day.

71. The method of claim 55, wherein said composition comprises autolysate in an isotonic medium.

72. The method of claim 55, wherein said composition is substantially free from non-suitable substances.

73. The method of claim 55, for improvement or normalizing of the immune system in subjects suffering from an immune system disorder including disorders as side effect caused by therapy of other diseases.

74. The method of claim 55, for use in treating domestic animals.
